(19) United States
(12) Patent Application Publication
(10) Pub. No.: US 2024/0368081 A1
(43) Pub. Date: Nov. 7, 2024

(54) PROCESS FOR THE PRODUCTION OF LEVETIRACETAM AND INTERMEDIATE THEREOF

(71) Applicant: Suzhou BrightHope Pharmatech Co., Ltd, Jiangsu Province (CN)

(72) Inventors: Songzhou Hu, Princeton, NJ (US); Lijun Deng, Hubei (CN); Wen Yu, Hubei (CN); Zhen Song, Shanghai (CN)

(21) Appl. No.: 18/143,790

(22) Filed: May 5, 2023

(51) Int. Cl. C07D 207/27

(52) U.S. Cl. CPC C07D 207/27; C07B 2200/07

(57) ABSTRACT

There is disclosed a process for the optical resolution of (RS)-α-ethyl-2-oxo-1-pyrrolidineacetic acid with a chiral amine and an optically inactive amine to form an ionic liquid in a solvent to produce (S)-α-ethyl-2-oxo-1-pyrrolidineacetic acid. The (S)-α-ethyl-2-oxo-1-pyrrolidineacetic acid is converted to (S)-α-ethyl-2-oxo-1-pyrrolidineacetamide.

### FIELD OF THE INVENTION

This invention relates to a process for the production of levetiracetam, more particularly, it relates to an improved process for the resolution of (RS)-α-ethyl-2-oxo-1-pyrrolidineacetic acid in the production of levetiracetam.

### BACKGROUND OF THE INVENTION

Levetiracetam is the S-enantiomer of etiracetam in a class of medications called anticonvulsants. It is used in combination with other medications to treat certain type of seizures in adults and children with epilepsy. Levetiracetam is chemically(S)-α-ethyl-2-oxo-1-pyrrolidineacetamide and has the following structure:

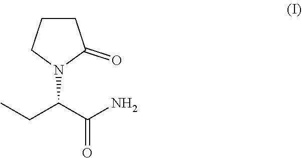

Although numerous methods have been developed for preparing levetiracetam, it is commercially produced by one of the two methods first disclosed in GB 1309692 and U.S. Pat. No. 4,696,943. In the first method, 2-pyrrolidinone and alkyl 2-halobutyrate are used as the starting materials in a process according to the following reaction scheme:

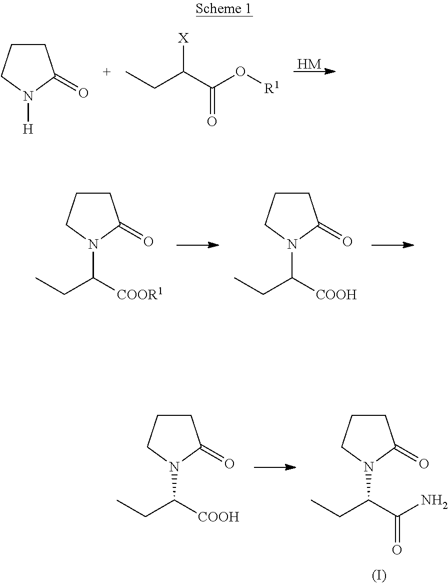

wherein X is halogen, HM is metal hydride, and R¹ is an alkyl group.

In the second method, L-2-aminobutanamide hydrochloride and alkyl 4-halobutyrate or 4-halobutyryl halide are used as the starting materials in a process according to the following reaction scheme:

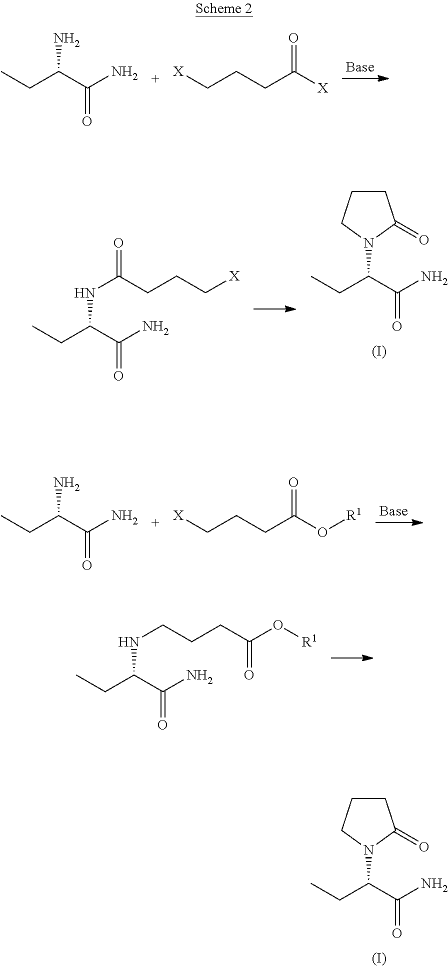

wherein X is halogen, R¹ is an alkyl group, and the base is an organic or inorganic base such as triethylamine or sodium carbonate.

There are inherent disadvantages in these two methods for the production of levetiracetam. In the process according to the first method, the metal hydride, which is required to deprotonate 2-pyrrolidinone, is dangerous to handle on a large scale and presents serious safety issues for the manufacturing plant. In addition, the alkyl 2-halobutyrate ester is not only unavailable commercially but also extremely obnoxious, posing environmental and occupational problems.

In the process according to the second method, just like the alkyl 2-halobutyate in the process of the first method, alkyl 4-halobutyrate and 4-halobutyryl halide are also extremely obnoxious, posing the same environmental and occupational problems. In addition, L-2-aminobutanamide hydrochloride is specifically produced for levetiracetam and as a result is costly. L-2-aminobutanamide hydrochloride is produced either from racemic 2-aminobutanamide by optical resolution or from L-2-aminobutyric acid, which is not one of the naturally occurring L-amino acids, but specifically produced for the purpose. Although there have been intensive efforts to improve the process for producing L-2-aminobutanamide hydrochloride and L-2-aminobutyric acid, they are still costly. In addition, during the cyclization under strongly basic condition, the product of levetiracetam is partially racemized or hydrolyzed.

U.S. Pat. No. 11,384,050 discloses an efficient process for the production of levetiracetam that has ameliorated much disadvantages of known processes. The process according this patent can construct necessary structure of a key intermediate of formula (II):

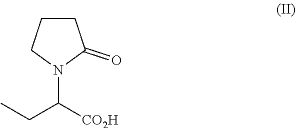

from readily available starting materials without isolating any intermediate.

In order to produce levetiracetam, it is necessary to resolve the racemic acid of formula (II) into the (S)-acid of formula (III) and the (R)-acid of formula (IV):

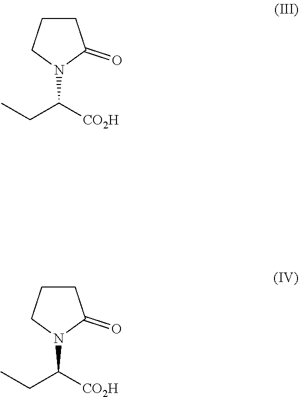

U.S. Pat. No. 4,696,943 discloses a method for the resolution of racemic acid of formula (II) by using an equimolar molar amount of R-(+)-1-phenylethylamine and triethylamine in benzene to obtain the (S)-acid of formula (III), which is subsequently converted to levetiracetam. It is apparently not desirable to use benzene as a solvent in the production of a pharmaceutical product.

U.S. Pat. No. 7,902,380 improves the method of U.S. Pat. No. 4,696,943 by performing the resolution in toluene by using a mixture of R-(+)-1-phenylethylamine and triethylamine and discloses a method for the racemization of the (R)-acid of formula (IV). The resolution was performed by recrystallizing the (S)-acid salt of R-(+)-1-phenylethylamine in toluene several times.

However, CN 101333180B found out that the crude (S)-acid salt of R-(+)-1-phenylethylamine has to be recrystallized five times in benzene as disclosed in U.S. Pat. No. 4,696,943 and four times in toluene as disclosed in U.S. Pat. No. 7,902,380, in order to obtain the (S)-acid of formula (II) with required optical purity to prepare levetiracetam.

CN 101333180B further discloses an improved method for the resolution of racemic acid of formula (II) by performing the resolution in ketones and esters as solvent and by using a mixture of R-(+)-1-phenylethylamine and triethylamine. However, the disclosed solvents are not suitable for industrial use, since ketones and esters are unstable and reactive towards the resolving agent of R-(+)-1-phenylethylamine.

It is an object of the present invention to overcome these disadvantages for the resolution of the racemic acid of formula (II). It is another object to disclose an economically advantageous process that can be used to produce the (S)-acid of formula (III) in high yield, in high optical purity, and in fewer steps for the production of levetiracetam.

### SUMMARY OF THE INVENTION

The present invention discloses an improved process for the production of levetiracetam, more specifically, it discloses an improved process for the resolution of racemic acid of formula (II) in the presence of an ionic liquid.

### DETAILED DESCRIPTION OF THE INVENTION

The present invention discloses an improved process for the production of levetiracetam, more particularly, it relates to an improved process for the resolution of the (RS)-acid of formula (II) to produce the (S)-acid of formula (III):

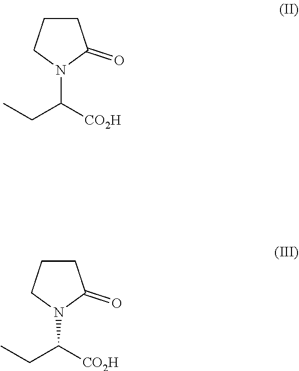

The (S)-acid of formula (III) is then converted to levetiracetam.

It has been found that an impurity was formed during the resolution of the racemic acid of formula (II) with R-(+)-1-phenylethylamine in toluene. LC-MS (liquid chromatography-mass spectrometry) analysis of this impurity shows a M⁺¹ peak at 275. Hence, this impurity is likely an amide of the following structure:

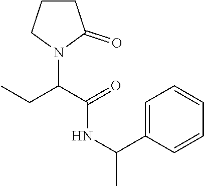 [SMILES: CCC(C(=O)NC(C)c1ccccc1)N1CCCC1=O]

It has further been found that the formation of this amide is temperature-dependent. At a temperature of lower than 80° C., in particular lower than 70° C., this impurity was not observed to form. At a refluxing temperature of toluene, the formation of this impurity is pronounced. In fact, extended heating can convert a majority of the acid and R-(+)-1-phenylethylamine into this impurity.

In order to avoid the formation of this impurity, it is necessary to perform all resolution steps at a temperature lower than 80° C., preferably, lower than 70° C. These steps include the formation of the (S)-acid salt of R-(+)-1-phenylethylamine, the purification of the (S)-acid salt, and the recovery of solvent containing the salt.

The present invention is accomplished by performing the resolution in a solvent containing an ionic liquid formed from the reaction of an optically inactive amine of formula R₁R₂R₃N with the racemic acid of formula (II) or the (S)-acid of formula (III) or the (R)-acid of formula (IV), wherein R₁, R₂, and R₃ are each independently hydrogen and alkyl group of C₁ to C₁₂ and wherein the total carbon number of R₁, R₂, and R₃ is at least 8. The invention provides an economically advantageous process that can obtain the (S)-acid of formula (III) in high yield, in high optical purity, and in fewer steps. The advantages of the present invention become apparent from the following description.

A suitable chiral amine resolving agent can be selected from those listed in David Kozma, CRC Handbook of Optical Resolution via Diastereomeric Salt Formation, 2002, CRC Press, which is incorporated herein in its entirety. Preferably, the chiral amine is R-(+)-1-phenylethylamine or dehydroabietylamine.

Suitable solvents for the resolution are aromatics, alkyl alcohols of C₄-C₈, and their mixture thereof. Suitable aromatics are selected from the group consisting of toluene, p-xylene, m-xylene, o-xylene, mixed xylenes, ethylbenzene, cumene, pseudocumene, chlorobenzene, dichlorobenzene, fluorobenzene, trifluoromethyl benzene, trimethylbenzenes, and a mixture thereof. Preferably, the aromatics are toluene and xylenes.

Suitable alkyl alcohols of C₄-C₈ are selected from the group consisting of n-butanol, isobutanol, 2-butanol, n-pentanol, cyclopentanol, isoamyl alcohol, hexanol, cyclohexanol, heptanol, octanol, 2-ethylhexanol, 2-octanol, and a mixture thereof. Preferably, the alcohol is n-butanol. More preferably, the alcohol is cyclohexanol.

Suitable optically inactive amines are selected from the group consisting of tripropylamine, tributylamine, N-methylmorpholine, N-methylpiperidine, N,N-dimethylbenzylamine, N,N-diethylbenzylamine, diisopropylethylamine, and a mixture thereof. Preferably, the optically inactive amine is tripropylamine or tributylamine. It has been found that these amines form an ionic liquid with the racemic acid of formula (II), the (S)-acid of formula (III), and the (R)-acid of formula (IV).

The process according to the present invention consists of mixing the racemic acid of formula (II), a chiral amine, and an optically inactive amine in a solvent. In the process according to the present invention, an optically inactive amine is used to form an ionic liquid with the racemic acid, or the (S)-acid of formula (III), or the (R)-acid of formula (IV) in situ. In addition, the use of these optically inactive amine can reduce the amount of chiral amine used in the resolution. The molar ratio of chiral amine to optically inactive amine can be from 0.1:10 to 10:0.1; preferably from 0.3:0.7 to 0.7:0.3; more preferably from 0.4:0.6 to 0.6:0.4; and most preferably from 0.45:0.55 to 0.55:0.45.

After the mixing of the racemic acid of formula (II), R-(+)-1-phenylethylamine, and an optically inactive amine, a solid suspension of the (S)-acid salt of R-(+)-1-phenylethylamine is precipitated immediately. This crystalline suspension may be filtrated to obtain the product. Preferably, this crystalline suspension is stirred and heated to a temperature lower than 80° C., preferably lower than 70° C. After cooling, the crystalline suspension is then filtrated to separate a crude salt of the (S)-acid and R-(+)-1-phenylethylamine and a mother liquor solution.

The mother liquor solution has been found to comprise predominantly the (R)-acid salt of optically inactive amine, and a small amount of the (R)-acid salt of R-(+)-1-phenylethylamine. It has been found that these salts can be readily separated from the mother liquor solution by extracting with water. The organic phase is the recovered solvent, which may be distilled for recycling use. Preferably, this recovered solvent can be recycled for the resolution without further purification. Therefore, distillation of the solvent may not be necessary in the process according to the present invention. This is a distinct advantage for the process according to the present invention.

Another added advantage of the process according to the present invention is the high yield recovery of the mixed solvents, because these solvents are nearly insoluble in water.

The aqueous phase has been found to comprise predominantly the (R)-acid salt of the optically inert amine. To this aqueous solution is added an alkali hydroxide to release the chiral amine and the optically inert amine. These amines can be recovered for reuse. The alkali salt of predominantly the (R)-acid can be used to produce racemic acid of formula (II). The alkali is lithium, sodium, potassium, and a mixture thereof.

The solid (S)-acid salt of R-(+)-1-phenylethylamine has been found to comprise 80-92% (S)-acid and 20-8% (R)-acid. In order to produce the (S)-acid of formula (II) to be suitable for the production of levetiracetam, this crude solid must be purified in a suitable solvent. Preferably, the same solvent is used as in the resolution stage to simplify the process.

While it is possible to recrystallize the crude (S)-acid salt in a suitable solvent, the temperature required to dissolve the crude (S)-acid salt is much higher than 80° C. and the formation of the amide impurity becomes significant. It has been gratifying to find that the crude (S)-acid salt can be optically enriched just by suspending it in a solvent containing an ionic liquid formed by the reaction of either racemic acid of formula (II) or the (S)-acid of formula (III) with an optically inactive amine at a temperature from 50° C. to 80° C., preferably from 60° C. to 70° C. At this temperature, the formation of the amide impurity is negligible. When the (S)-acid salt of R-(+)-1-phenylethylamine is used as the ionic liquid in a suitable solvent, a crude (S)-acid salt can be enriched to more than 97% of(S)-acid, a purity required for the production of levetiracetam.

Preferably, the same optically inactive amine as used in the resolution stage is used in the preparation of the ionic liquid. More preferably, the (S)-acid salt of the same optically inactive amine is used as the ionic liquid to purify a crude (S)-acid salt of R-(+)-1-phenylethylamine. When (S)-acid of formula (III) is used, its optical purity can be from 50% to 98%, preferably from 70-90%, more preferably, from 75-85%.

It has been found that the crude (S)-acid salt of R-(+)-1-phenylethylamine can also be enriched in a mixed solvent containing the resolving agent of R-(+)-1-phenylethylamine or the optically inactive amine. This finding is important in a cyclic use of the mixed solvent without further purification. After the resolution, the mother liquor solution comprising aromatics and an alcohol of C₄-C₈ can be washed with water to remove dissolved salt to recover the solvent. This solvent can be used to recover the resolving agent of R-(+)-1-phenylethylamine by extraction from an aqueous solution. The resultant solution of R-(+)-1-phenylethylamine in the mixed solvent can be used directly for the purification of the crude (S)-acid salt and then for the resolution.

The (S)-acid salt can be decomposed to recover the chiral amine and to isolate the (S)-acid of formula (III). One method is to dissolve the (S)-acid salt in a solution of alkali hydroxide to release the chiral amine, which can be extracted with an extractant solvent. Suitable extractant solvent is water-insoluble solvent or a mixture of water-insoluble solvents. Preferably, the same solvent in the resolution step is used as the extractant solvent. After the chiral amine is recovered, the alkali solution is then acidified to precipitate the (S)-acid of formula (III). A suitable acid is selected from the group consisting of hydrochloric acid, hydrobromic acid, sulfuric acid, nitric acid, phosphoric acid, formic acid, acetic acid, propionic acid, glycolic acid, tartaric acid, malic acid, citric acid, alkyl sulfonic acid, aromatic sulfonic acid, and a mixture thereof.

Another method to decompose the (S)-acid salt is to add the salt to an acidic solution to precipitate the (S)-acid of formula (III). A suitable acid is selected from the group consisting of hydrochloric acid, hydrobromic acid, sulfuric acid, nitric acid, phosphoric acid, formic acid, acetic acid, propionic acid, glycolic acid, tartaric acid, malic acid, citric acid, alkyl sulfonic acid, aromatic sulfonic acid, and a mixture thereof. After the separation of the (S)-acid of formula (III), the chiral amine can be isolated by adding an alkali hydroxide to the solution.

The (S)-acid of formula (III), produced in the process according to the present invention, can be converted to levetiracetam of formula (I) by methods known to one skilled in the art.

The process according to the present invention can be carried out discontinuously, semi-continuously, or continuously.

### EXAMPLES

The following examples will illustrate the practice of this invention but are not intended to limit its scope.

### Comparative Example 1

This example demonstrated the formation of an amide impurity in refluxing toluene.

To a round bottom flask were added 58.4 g of the (S)-acid salt of R-(+)-1-phenylethylamine and 240 mL of toluene. Upon heating to 110° C., a clear solution was obtained. This clear solution was stirred at this temperature for 4 hours. High performance liquid chromatography showed the formation of an impurity and the ratio of the integration area for the (S)-acid and the impurity was 52.7%:47.3%. Upon cooling to the 20° C., (S)-acid salt of R-(+)-1-phenylethylamine crystallized. After filtration and washing with toluene, this precipitated product did not contain detectable impurity. In the mother liquor solution, the ratio of the integration area for the (S)-acid and the impurity was 39.5%:60.6%. High performance liquid chromatography-mass spectrometry showed a M⁺¹ peak at 275.

### Comparative Example 2

This example demonstrated that it was not possible to purify a crude (S)-acid salt of R-(+)-1-phenylethylamine to desired optical purity in toluene at a temperature of 70° C.

To a round bottom flask were added 600 mL of toluene and 150 g of a crude (S)-acid salt of R-(+)-1-phenylethylamine with (S)-acid content of 84%. The suspension was stirred at 70° C. for 1 hour, and then slowed cooled to 30° C. and maintained at this temperature for another hour. The suspension was then filtrated and washed with toluene. The product contained 92.7% of the (S)-acid and 7.3% of the (R)-acid. The ratio of (R)-acid and (S)-acid in the mother liquor solution was 83.5%: 16.5%.

The filtration wet cake was suspended in 570 mL of toluene and stirred at 70° C. for 1 hour, and then slowed cooled to 30° C. and maintained at this temperature for another hour. The suspension was then filtrated and washed with toluene. The product contained 94.1% of the (S)-acid and 5.9% of the (R)-acid. The ratio of (R)-acid and (S)-acid in the mother liquor solution was 71.4%:28.6%.

### Example 1

To a round bottom flask were added 230 g of xylene, 36.3 g of R-(+)-1-phenylethylamine, and 50.0 g of tripropylamine, and 111.0 g of racemic acid of formula (II). The solution becomes a crystalline suspension immediately. The suspension was heat to 70° C. for 30 minutes and cooled to room temperature. The suspension was filtrated and washed with xylene. After drying, the product weighted 79.6 g. The ratio of the (S)-acid and the (R)-acid in the wet cake were 84.5%:15.5%.

### Example 2

To a round bottom flask were added 85 mL of xylene, 8.6 g of the (S)-acid and 7.2 g of tripropylamine, followed by 13.0 g of a crude (S)-acid salt of R-(+)-1-phenylethylamine. The suspension was stirred and heated to 85° C. to obtain a clear solution and then cooled to 16° C. to form a crystalline suspension. The suspension was filtrated and washed with xylene. The ratio of the (S)-acid and the (R)-acid in the wet cake were 96.8%:3.2%, while the ratio of the (S)-acid and the (R)-acid in the mother liquor solution was 30.9%:69.1%.

### Example 3

To a round bottom flask were added 50 mL of toluene, 3.6 g of tripropylamine, 7.3 g of racemic acid of formula (II), followed by 10 g of a crude (S)-acid salt of R-(+)-1-phenylethylamine. The suspension was heated briefly to 90° C. and quickly cooled to 16° C. to obtain a crystalline suspension. The suspension was filtrated and washed with 50 mL of toluene. The ratio of the (S)-acid and the (R)-acid in the wet cake were 98.4%: 1.6%, while the ratio of the (S)-acid and the (R)-acid in the mother liquor solution was 70%:30%.

### Example 4

To a round bottom flask were added 70 mL of a mixed solvent comprising 10% of cyclohexanol and 90% of o-xylene by volume, 7.2 g of R-(+)-1-phenylethylamine, 7.8 g of tripropylamine, and 17.1 g of the racemic acid of formula (II). A crystalline suspension was formed immediately. The suspension was stirred at 70° C. for 30 minutes and cooled to 15° C. in 40 minutes. The suspension was filtrated and washed with 30 mL of the same mixed solvent. The ratio of the (S)-acid and the (R)-acid in the wet cake were 84.6%:15.4%, while the ratio of the (S)-acid and the (R)-acid in the mother liquor solution was 18.4%:81.6%.

### Example 5

To a flask were added 1.3 L of anhydrous methanol, 6.8 g of sulfuric acid, and 240 g of the (S)-acid of formula (III). After the solution was refluxed for 3 hours, methanol was removed to yield a solution of the methyl ester. This solution was cooled to 5° C. and added to 448 g of 26% aqueous ammonia solution. The solution was stirred at 10° C. for 15 hours. The solution was concentrated under vacuum to 330 g and cooled to 5° C. to obtain a crystalline suspension of levetiracetam. The crystalline solid was separated by filtration.

147 g of the crystalline solid was dissolved in 250 mL of acetone and warmed to 60° C. for 1 hour. After cooling to 20° C., the crystalline suspension was filtrated to yield 115 g of levetiracetam after drying. The purity of the product is 99.9% by HPLC and the optical purity of the product is 99.99% by chiral analysis.

### Example 6

To a round bottom flask were added 3.45 g of dehydroabietylamine acetate, 3.42 g of the racemic acid of formula (II), 2.9 g of tripropylamine, and 50 g of toluene/butanol. The mixture was stirred and heated to 70° C. for 15 minutes and then cooled to 17° C. to obtain a crystalline suspension. The crystalline solid was filtrated and washed with a mixed solvent of toluene-butanol. The ratio of the (S)-acid and the (R)-acid in the wet cake were 95.2%:4.8%, while the ratio of the (S)-acid and the (R)-acid in the mother liquor solution was 43.6%:56.4%.

It will be understood that the foregoing examples, explanation, and drawings are for illustrative purposes only and that in view of the instant disclosure various modifications of the present invention will be self-evident to those skilled in the art. Such modifications are to be included within the spirit and purview of this application and the scope of the appended claims.

What is claimed is:

1. A process for the production of(S)-α-ethyl-2-oxo-1-pyrrolidineacetamide of formula (I), 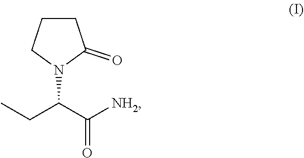
comprising:
(a) resolving (RS)-α-ethyl-2-oxo-1-pyrrolidineacetic acid of formula (II): 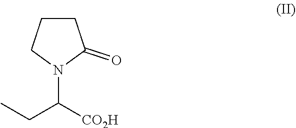
with a chiral amine and an optically inactive amine of R₁R₂R₃N to prepare (S)-α-ethyl-2-oxo-1-pyrrolidineacetic acid of formula (III): 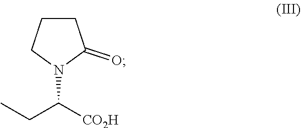
wherein R₁, R₂, and R₃ are each independently alkyl group of C₁-C₁₂ and the total carbon number of R₁, R₂, and R₃ is at least 8; and
(b) converting the (S)-α-ethyl-2-oxo-1-pyrrolidineacetic acid of formula (III) to(S)-α-ethyl-2-oxo-1-pyrrolidineacetamide of formula (I).

2. The process according to claim 1, wherein the chiral amine is R-(+)-1-phenylethylamine.

3. The process according to claim 1, wherein the chiral amine is dehydroabietylamine.

4. The process according to claim 1, wherein the optically inactive amine is selected from the group consisting of tripropylamine, tributylamine, N-methylmorpholine, N-methylpiperidine, N,N-dimethylbenzylamine, N,N-diethylbenzylamine, diisopropylethylamine, and a mixture thereof.

5. The process according to claim 1, wherein the optically inactive amine is tripropylamine.

6. The process according to claim 1, wherein the optically inactive amine is tributylamine.
